(19) United States
(12) Patent Application Publication
(10) Pub. No.: US 2013/0067707 A1
(43) Pub. Date: Mar. 21, 2013

(54) METHOD FOR WRAPPING A BODY IN FIBER MATERIAL

(71) Applicant: Rudolf Kaiser, Lauchringen (DE)

(72) Inventor: Rudolf Kaiser, Lauchringen (DE)

(21) Appl. No.: 13/701,753

(22) PCT Filed: Jun. 6, 2011
(86) PCT No.: PCT/EP11/59294

§ 371 Date: Dec. 3, 2012

(30) Foreign Application Priority Data
Jun. 16, 2010 (EP) 10166154.4

(51) Int. Cl. A61F 13/15; A61F 13/20; A61H 21/00

(52) U.S. Cl. CPC A61F 13/15666; A61H 21/00; A61F 13/20
USPC 28/118; 53/461

(57) ABSTRACT

The disclosure solves the problem of wrapping a body, such as a vibration generator present in a capsule, in a fiber material in order to form a tampon (2), for example. The solution is that the fiber material is wound about the body.

The invention relates to a method for wrapping a body with fiber material.

>From references U.S. Pat. No. 5,782,779, U.S. Pat. No. 6,183,428, and US2007/0260210, tampon assemblies having a vibration element embedded in the tampon body are known in the art. Tampon assemblies of this kind are used against menstrual cramps. Regarding the embedding of the vibration element in the tampon body, it merely follows from the reference US2007/0260210 that the vibration element is contained in a capsule on the exterior of which retaining elements are arranged in order to prevent the capsule from being pulled out of the tampon body particularly when the assembly is withdrawn from the vagina. In this reference, the tampon body is described as a hollow body into which the capsule is apparently inserted. On one hand, the manufacture of a hollow body from fiber material is not easy, and on the other hand, the subsequent assembly with the capsule entails additional operations.

On the background of this prior art, it is the object of the invention to suggest a method that does not suffer from the aforementioned disadvantages and is simple, fast, and inexpensive to implement, in particular.

According to the invention, this object is achieved in that the fiber material is wound around the body.

This solution according to the invention offers the advantage that by winding the fiber material around the body, process steps for the separate manufacture of a hollow body from fiber material can be eliminated.

According to one embodiment of the method of the invention the fiber material is in the form of a fabric. A fabric is well suited for forming a wrapping.

The fiber material is preferably in the form of a strip. A strip is particularly well suited for forming a wrapping that is finally intended to form an absorbent body.

According to another embodiment the body is a preferably elongated capsule having a content. Regarding the content, diagnostic or therapeutic devices including devices for the delivery of active agents may be contemplated.

According to a further embodiment the content of the capsule includes an energy consuming device. Such energy consuming devices may e.g. be electrically, pneumatically, or hydraulically operated devices.

According to a further embodiment the energy consuming device is a vibration generator. As mentioned in the introduction, such an assembly may e.g. be used for influencing menstruation.

According to another embodiment the body is connected to a flexible pulling means that preferably comprises at least one conductor for energy. The pulling means may serve for withdrawing the assembly from a body orifice and the conductor for supplying a load with energy.

According to a further embodiment the body is retained in a fixture, and the fixture with the body retained therein and the fiber material are rotated relative to one another in order to form a winding. In this manner, several layers of the fiber material are built up around the body relatively easily and quickly.

According to a further embodiment the fixture is fork-shaped and has at least two fork prongs. These two prongs allow retaining the body.

According to a further embodiment the fiber material is placed between the body and at least one of the fork prongs before the rotating operation. The fiber material is thus retained relative to the body and can be wound up.

According to another embodiment the fiber material is placed between the body and at least two of the fork prongs before the rotating operation. In this manner the body is already encircled by fiber material on a part of its circumference before the rotating operation.

According to a further embodiment the fiber material is laid around the body in a loop and the flexible pulling means is guided over the loop in parallel to the body. The thus achieved deflection of the flexible pulling means results in an excellent pullout protection of the pulling means from the body and subsequently also of the body from the winding.

According to a further embodiment the body is inserted into the fixture, the fiber material is positioned next to the fixture axially adjacent to the body in the area of the pulling means, and the body is turned around about an axis extending perpendicularly to its longitudinal axis such that the body draws the fiber material into the fixture while forming the loop. The aforementioned deflection of the pulling means is thus achieved in a simple manner.

According to a further embodiment, after the rotating operation, the resulting winding is slipped off the fixture axially and introduced into a forming tool. In this forming tool the winding is given its final shape, e.g. the shape of a tampon. Preferably, the forming tool has an internal diameter that is smaller than the external diameter of the winding. Consequently, the fiber material is compressed in the forming tool.

According to a further embodiment the winding is provided with a rounded tip by means of the forming tool. This tip facilitates the insertion of the assembly e.g. into a body orifice.

Ultimately, according to a further embodiment, the forming tool is heated. In particular it is thus achieved that the winding maintains the deformed state.

Embodiments of the method according to the invention will now be explained using the example of a tampon assembly and with reference to the appended drawings showing:

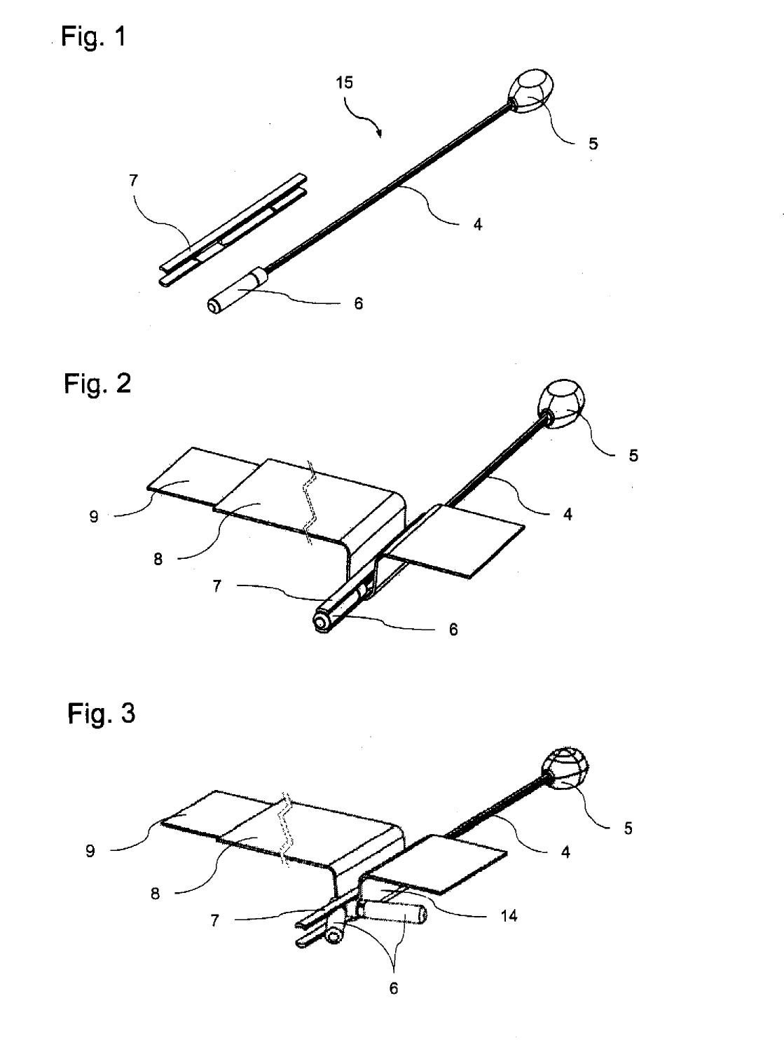

FIG. 1 a perspective view of a pre-mounted assembly containing a capsule and of a fixture in the initial position prior to being joined by means of fiber material,

FIG. 2 the placement in the fixture and a surrounding fiber strip,

FIG. 3 the capsule being turned around,

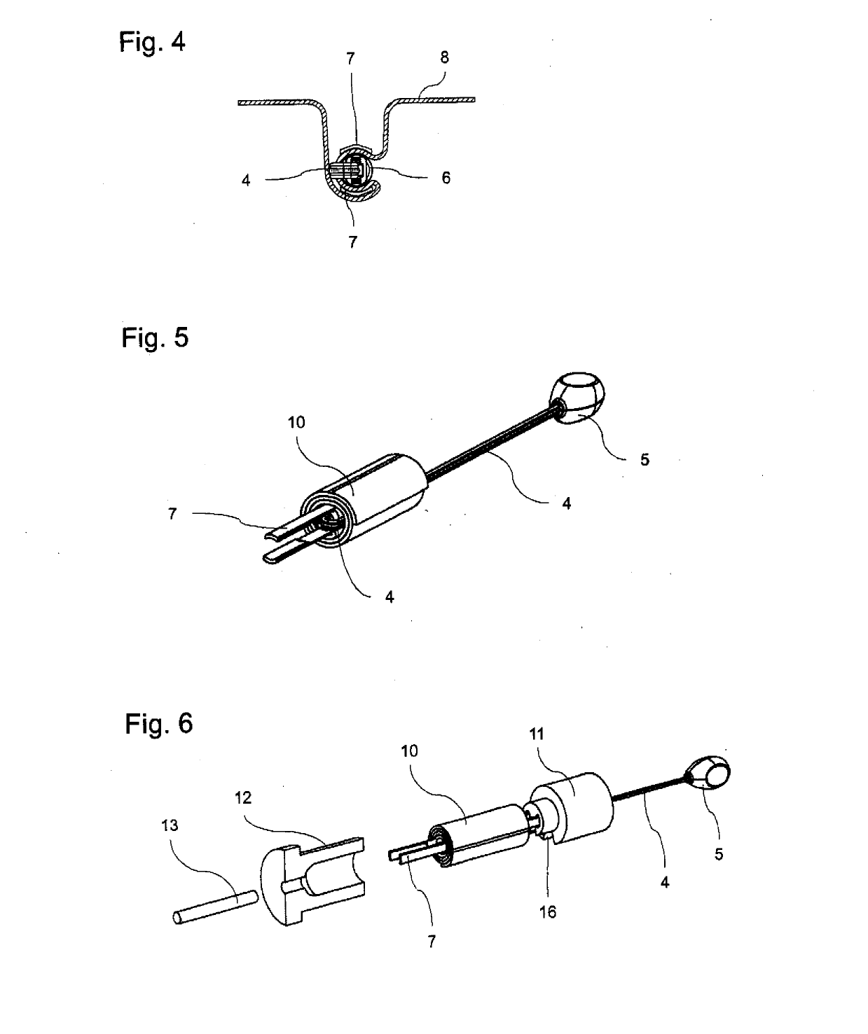

FIG. 4 a top view of the assembly and of the fixture prior to the winding operation,

FIG. 5 a perspective view of the assembly and of the fixture after the winding operation,

FIG. 6 the assembly with further tools,

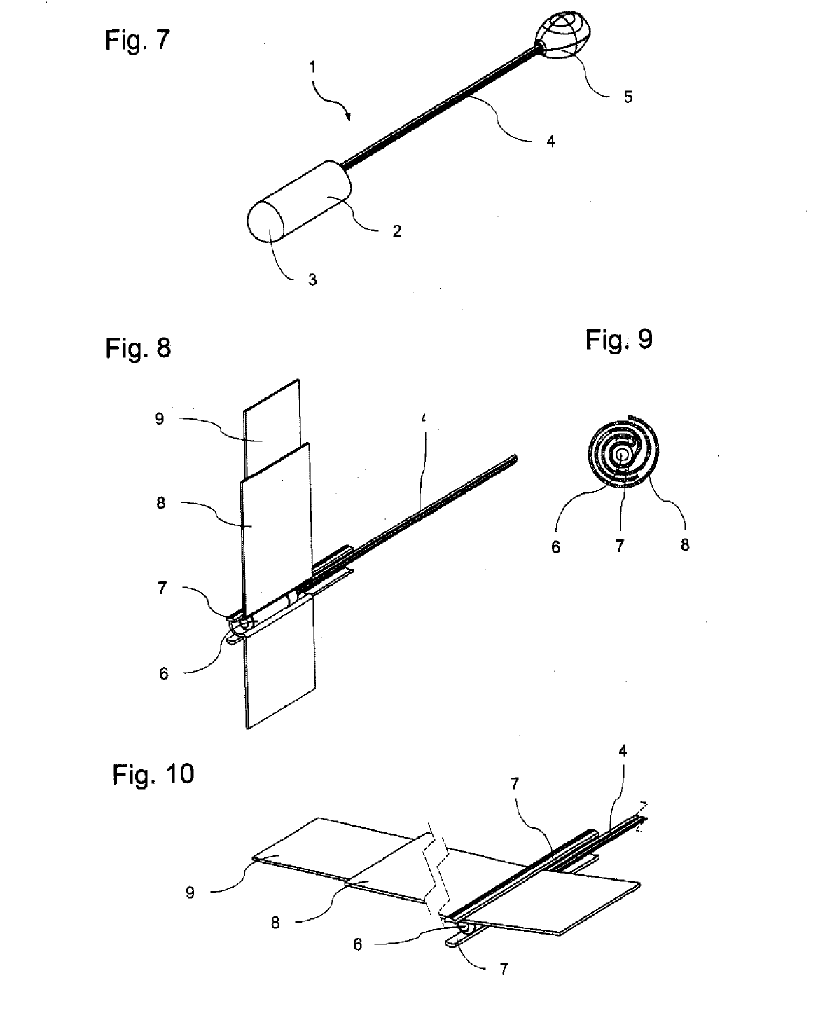

FIG. 7 a finished tampon assembly,

FIG. 8 an alternative arrangement of the components prior to the winding operation,

FIG. 9 based on FIG. 8, a front view of the components after the winding operation,

FIG. 10 a further alternative arrangement of the components prior to the winding operation,

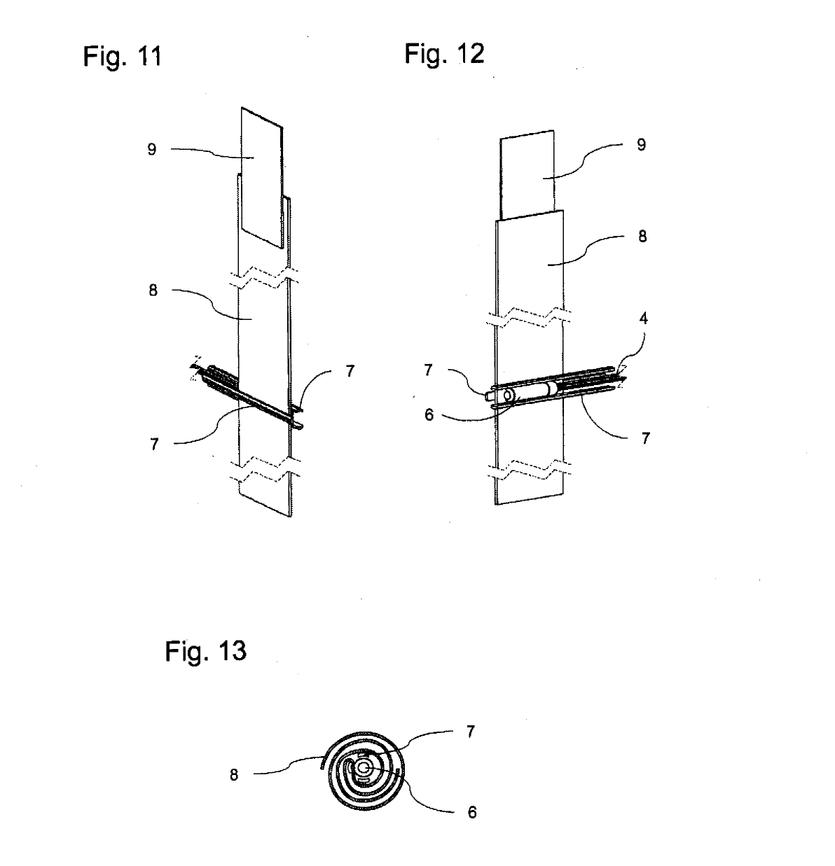

FIGS. 11 and 12 a further alternative arrangement of the components prior to the winding operation, and

FIG. 13 based on FIGS. 11 and 12, a front view of the components after the winding operation.

Exemplary embodiments of the method of the invention will be described hereinafter with reference to the manufacture of a tampon assembly 1 (see FIG. 7) comprising a vibration generator. However, the invention is not limited to the manufacture of such tampon assemblies. On the contrary, the invention is applicable to the manufacture of bodies of all kinds that are wrapped with fiber material. Furthermore, the body may e.g. comprise a heating element or sensors or may be designed as a capsule containing a medicine.

FIG. 1 shows a pre-mounted assembly 15 consisting of a capsule 6 containing a vibration generator, a cable 4 serving for current conduction and as a withdrawal means, and an energy supply and control unit 5. It is the object of the method described hereinafter to manufacture a tampon 2 (FIG. 7) that surrounds capsule 6 at least partly. Reference numeral 7 designates a fork-shaped fixture having two fork prongs that is intended to receive and retain capsule 6. Starting from the position illustrated in FIG. 1, capsule 6 is preferably laterally inserted between the fork prongs of fixture 7.

FIG. 2 shows fixture 7 with capsule 6 inserted therein. At the cable-side end of the capsule, a fiber strip 8 is laid around fixture 7 in a loop such that capsule 6 is not covered by the fiber strip. A fleece strip 9 is connected to fiber strip 8 and is intended to at least partly cover the surface of the finished tampon at the end of the process.

In FIG. 3 it is illustrated how the capsule is turned around in fixture 7. Two intermediate positions of capsule 6 are shown in dotted lines. As the capsule is further turned around from the position shown in FIG. 3 in the counterclockwise direction, it will enter into contact with the outer surface of the loop in the area designated by reference numeral 14 and in its further movement will draw fiber strip 8 between the fork prongs of fixture 7. When the turnaround is completed, the capsule is rotated 180 degrees with respect to FIG. 2 and positioned in fixture 7 coaxially, and fiber strip 8 is clamped between capsule 6 and the two fork prongs of fixture 7 and forms a loop whereas cable 4 extends outside the loop in parallel to capsule 6. This process step results in two substantial advantages. Firstly, cable 4 is deflected at the end of capsule 6 whereby the pullout protection of the cable 4 from capsule 6 is increased. Secondly, from the end of the capsule, cable 4 runs around the longitudinal edge of fiber strip 8, thereby ultimately increasing the pullout protection of capsule 6 from the tampon.

When capsule 6 is positioned coaxially in fixture 7 at the end of the process step according to FIG. 3, the latter is rotated in the counterclockwise direction relative to the Figure together with cable 4 and energy supply and control unit 5. At the same time, fiber strip 8 is guided so as to be wound around fixture 7 and capsule 6 retained therein. FIG. 4 shows a top view of assembly 1 and fixture 7 based on the illustration in FIG. 3, after the turnaround is completed. Capsule 6 is encircled by fiber strip 8 and located between the prongs of fixture 7. By rotating fixture 7 about its longitudinal axis, the two free ends of fiber strip 8 are now wound outside around the two fork prongs. It is also seen in the Figure that cable 4 is guided over the outside of the first layer of fiber strip 8 and subsequently covered with additional layers of fiber strip 8. With suitable means it is also possible to wind fiber strip 8 around fixture 7 with capsule 6 retained therein while fixture 7 remains stationary. In principle, a combined counter-rotation of fixture 7 and fiber strip 8 is also possible.

FIG. 5 shows the winding 10 created by the described rotational movement, and fixture 7. Here also it is seen that cable 4 runs from the end of capsule 6 around the latter such that fiber strip 8 lies between capsule 6 and cable 4 in this area.

FIG. 6 shows winding 10 before the following process step that comprises slipping off winding 10 from fixture 7 and compacting and shaping the winding to form a tampon 2 (FIG. 7). To slip off winding 10, a slip-off tool 11 is provided that is longitudinally displaceable on fixture 7 and has a longitudinal slot 16 to allow the subsequent removal of cable 4. By a longitudinal movement of slip-off tool 11 relative to fixture 7, winding 10 is slipped off from fixture 7 and pushed into a forming tool 12 having an opening whose internal diameter is smaller than the external diameter of winding 10 and substantially corresponds to the diameter of the finished tampon 2 (see FIG. 7). Due to the aforementioned difference in diameter, the winding is radially compressed. The end of the opening in forming tool 12 is rounded and preferably shaped hemispherically. By means of slip-off tool 11, winding 10 is compacted also radially so that the finished tampon is provided with a rounded head 3 (FIG. 7) at its insertion end. Forming tool 12 is heated, e.g. to 100° C., and the compressed winding 10 remains in forming tool 12 for a specified time, e.g. 20 seconds. As a result, the finished tampon will remain dimensionally stable after being removed from forming tool 12. Moreover, the heat may serve to produce a partial welding of fleece strip 9, for which purpose the latter preferably consists of a fusible material. To remove tampon 2, it is pushed out of forming tool 12 by means of an ejector 13.

FIG. 7 shows the finished tampon assembly 1 with tampon 2, rounded head 3, cable 4, and energy supply and control unit 5.

FIGS. 8 and 9 illustrate that capsule 6 may alternatively be wrapped in fiber strip 8 without being previously turned around in fixture 7. To this end, capsule 6 and fixture 7 are first positioned adjacent to each other with parallel longitudinal axes and with fiber strip 8 between them. Then, capsule 6 and/or fixture 7 is/are parallelly displaced so that capsule 6 is finally located between the prongs of fixture 7, as shown in FIG. 8, while a layer of fiber strip 8 extends between the capsule and each prong of fixture 7. FIG. 9 shows a top view of capsule 6 and fixture 7 based on the illustration in FIG. 8 after the completed winding operation.

Another possibility of bringing capsule 6, fixture 7, and fiber strip 8 into a starting position for the winding operation is illustrated in FIG. 10. Here, fiber strip 8 is preferably first placed between the prongs of fixture 7 as illustrated, and then capsule 6 is laterally introduced into fixture 7 such that it is in direct contact at the bottom of the Figure with one prong of fixture 7 and at the top of the Figure a layer of fiber strip 8 is present between capsule 6 and the upper prong of fixture 7.

Ultimately, FIGS. 11 to 13 show yet another possibility of bringing capsule 6, fixture 7, and fiber strip 8 into a starting position for the winding operation. In this case, in contrast to the previously described methods, fixture 7 has three prongs. In this manner it is possible to place capsule 6 and fiber strip between the fork prongs in any order. For example, as shown in FIG. 11, fiber strip 8 may be inserted between the prong illustrated on the left of this Figure and the two other prongs of fixture 7, and capsule 6 is introduced between the two other prongs, as seen in FIG. 12. FIG. 13 shows the course of fiber strip 8 relative to capsule 6 and fixture 7 after the winding operation in a front view.

### LIST OF REFERENCE NUMERALS

1 tampon assembly
2 tampon
3 head
4 cable
5 energy supply and control unit
6 vibration drive
7 fixture
8 fiber strip
9 fleece strip
10 winding
11 slip-off tool
12 forming tool
13 ejector
14 outer surface
15 pre-mounted assembly
16 longitudinal slot

1. Method for wrapping a body with fiber material, wherein the body is an elongated capsule having a content, the method comprising winding the fiber material around the body.

2. The method according to claim 1, wherein the fiber material is in the form of a fabric.

3. The method according to claim 2, wherein the fiber material is in the form of a strip.

4. The method according to claim 1, wherein the content of the capsule includes an energy consuming device.

5. The method according to claim 4, wherein the energy consuming device is a vibration generator.

6. The method according to claim 1, further comprising connecting the body to a flexible pulling means that comprises at least one conductor for energy.

7. The method according to claim 1, further comprising retaining the body in a fixture and rotating the fixture with the body retained therein relative to the fiber material in order to form a winding.

8. The method according to claim 7, wherein the fixture is fork-shaped and has at least two fork prongs.

9. The method according to claim 8, further comprising placing the fiber material between the body and at least one of the fork prongs before the rotating operation.

10. The method according to claim 9, further comprising placing the fiber material between the body and at least two of the fork prongs before the rotating operation.

11. The method according to claim 6, further comprising laying the fiber material around the body in a loop and guiding the flexible pulling means over the loop in parallel to the body, and placing the fiber material between the body and at least two of the fork prongs before the rotating operation.

12. The method according to claim 11, further comprising inserting the body into the fixture, positioning the fiber material next to the fixture axially adjacent to the body in the area of the pulling means, and turning the body around an axis extending perpendicularly to its longitudinal axis such that the body draws the fiber material into the fixture while forming the loop.

13. The method according to claim 7, further comprising after the rotating operation, slipping the resulting winding off the fixture axially and introducing the fixture into a forming tool.

14. The method according to claim 13, wherein the forming tool has an internal diameter that is smaller than the external diameter of the winding.

15. The method according to claim 13, further comprising providing the winding with a rounded tip by means of the forming tool.

16. The method according to claim 13, further comprising heating the forming tool.
